(19) United States
(12) Patent Application Publication
(10) Pub. No.: US 2008/0027544 A1
(43) Pub. Date: Jan. 31, 2008

(54) Instruments and techniques for engaging spinal implants for insertion into a spinal space

(75) Inventor: Anthony J. Melkent, Memphis, TN (US)

Correspondence Address: KRIEG DEVAULT LLP, ONE INDIANA SQUARE, SUITE 2800, INDIANAPOLIS, IN, 46204-2709, US

(73) Assignee: Warsaw Orthopedic Inc.

(21) Appl. No.: 11/495,981

(22) Filed: Jul. 28, 2006

(51) Int. Cl. A61F 2/44

(52) U.S. Cl. 623/17.11

(57) ABSTRACT

Instruments, implants and methods are provided for positioning spinal implants in a space between vertebrae. The instruments provide a low profile engagement with the implants and facilitate insertion while minimizing tissue retraction and exposure of the tissue and neural elements to the instrumentation in the approach to the space.

### BACKGROUND

Normal intervertebral discs between endplates of adjacent vertebrae distribute forces between the vertebrae and cushion vertebral bodies. The spinal discs may be displaced or damaged due to trauma, disease or aging. A herniated or ruptured annulus fibrosis may result in nerve damage, pain, numbness, muscle weakness, and even paralysis. Furthermore, as a result of the normal aging processes, discs dehydrate and harden, thereby reducing the disc space height and producing instability of the spine and decreased mobility. Most surgical corrections of a disc space include a discectomy, which can be followed by restoration of normal disc space height and bony fusion of the adjacent vertebrae to maintain the disc space height.

Other procedures can involve removal of one or more vertebral bodies as a result of trauma, disease or other condition. An implant can be positioned between intact vertebrae to provide support until fusion of the affected spinal column segment is attained.

Access to a damaged disc space or to a corpectomy location may be accomplished from several approaches to the spine. One approach is to gain access to the anterior portion of the spine through a patient's abdomen. A posterior or lateral approach may also be utilized. Postero-lateral, antero-lateral and oblique approaches to the spinal column have also been employed to insert implants. Whatever the approach, there remains a need for improved instruments and techniques for inserting spinal implants that minimize intrusion of the inserter instrument into the anatomy adjacent the implantation location.

### SUMMARY

There are provided instruments, implants and methods useful for providing assemblies to insert implants in a space between vertebrae that include supporting the implant on the instrument in a first mode that allows the implant and instrument to move relative to one another in at least plane and rigidly securing the implant to the instrument in a second mode.

According to one aspect, there is provided an assembly for spinal stabilization procedures. The assembly includes an implant including a body with a first receptacle and a second receptacle. The assembly also includes an inserter instrument engaged to the first and second receptacles of the implant. The inserter instrument includes a proximal actuator assembly extending along a longitudinal axis and an engaging assembly at a distal end of the proximal actuator assembly. The engaging assembly includes a support member and a coupling member. The coupling member is movable relative to the support member along the longitudinal axis. The support member includes a cylindrical body extending transversely to the longitudinal axis and the coupling member includes a cylindrical body extending along the longitudinal axis. The first receptacle includes a cylindrical size and shape to receive the first support member and the second receptacle includes a cylindrical size and shape to receive the second support member.

According to another aspect, an assembly for spinal stabilization procedures comprises an implant including a body with a receptacle and an inserter instrument. The inserter instrument includes a proximal actuator assembly extending along a longitudinal axis and an engaging assembly at a distal end of the proximal actuator assembly. The engaging assembly includes a coupling member and an actuating member extending through the coupling member. The actuating member is movable upon actuation of the actuator assembly to expand the coupling member in the receptacle and engage the implant to the engaging assembly.

According to a further aspect, an assembly for spinal stabilization procedures comprises an implant including a body with a receptacle and a post in the receptacle and an inserter instrument. The inserter instrument comprises a proximal actuator assembly extending along a longitudinal axis and an engaging assembly at a distal end of the proximal actuator assembly. The engaging assembly includes first and second coupling members, at least one of which is movable relative to the other. Distal ends of the coupling members are spaced from one another in an open position for positioning around the post and movable with the actuator assembly to a closed position wherein the first and second coupling members are clampingly engaged to the post.

These and other aspects will be further discussed below with reference to the drawings.

### BRIEF DESCRIPTION OF THE DRAWINGS

FIG. 1 is a plan view of an implant and an inserter instrument spaced from the implant.

FIG. 2 is an elevation view of a distal portion of the inserter instrument and the implant of FIG. 1 in an assembled fashion and with the implant in a space between vertebrae.

FIG. 2A shows another embodiment support member of the inserter instrument of FIG. 1.

FIG. 3 is a plan view of another embodiment implant and a distal portion of another embodiment inserter instrument spaced from the implant.

FIG. 4 is a sectional view of the distal portion of the inserter instrument of FIG. 3.

FIG. 4A is a perspective view of another embodiment distal portion of an inserter instrument.

FIG. 5 is an elevation view of an actuator assembly of the inserter instrument of FIG. 3 with an expandable portion of the engaging assembly of the inserter instrument removed from the actuating member of the actuator assembly.

FIG. 6 is a plan view of another embodiment implant and a distal portion of another embodiment inserter instrument spaced from the implant.

FIG. 7 is an elevation view of the implant and the distal portion of the inserter instrument of FIG. 6.

FIG. 8 is a plan view of the distal portion of the inserter instrument of FIG. 6 in an open position.

FIG. 9 is a plan view of the distal portion of the inserter instrument of FIG. 6 in a closed position.

FIG. 10 is an elevation of one embodiment actuator assembly of the inserter instrument of FIG. 6 with the distal engaging assembly removed.

### DETAILED DESCRIPTION OF THE ILLUSTRATED EMBODIMENTS

For the purposes of promoting an understanding of the principles of the present invention, reference will now be made to the embodiments illustrated in the drawings, and specific language will be used to describe the same. It will nevertheless be understood that no limitation of the scope of the invention is intended thereby. Any alterations and further modification in the described processes, systems, or devices, and any further applications of the principles of the invention as described herein are contemplated as would normally occur to one skilled in the art to which the invention relates.

Instruments, implants and techniques provide assemblies for implant insertion into a space between vertebrae defined by a spinal disc or one or more vertebrae and associated spinal discs. The instruments and implants can be employed in any approach to the space, including anterior, lateral, posterior, postero-lateral and antero-lateral approaches. The instruments and implants facilitate positioning and manipulating the implant in the space while minimizing intrusion of the instrument into the space. The inserter instruments provide a low profile engagement with the implant to minimize the footprint of the assembly and minimize exposure and retraction of tissue and neural elements to accommodate implant insertion.

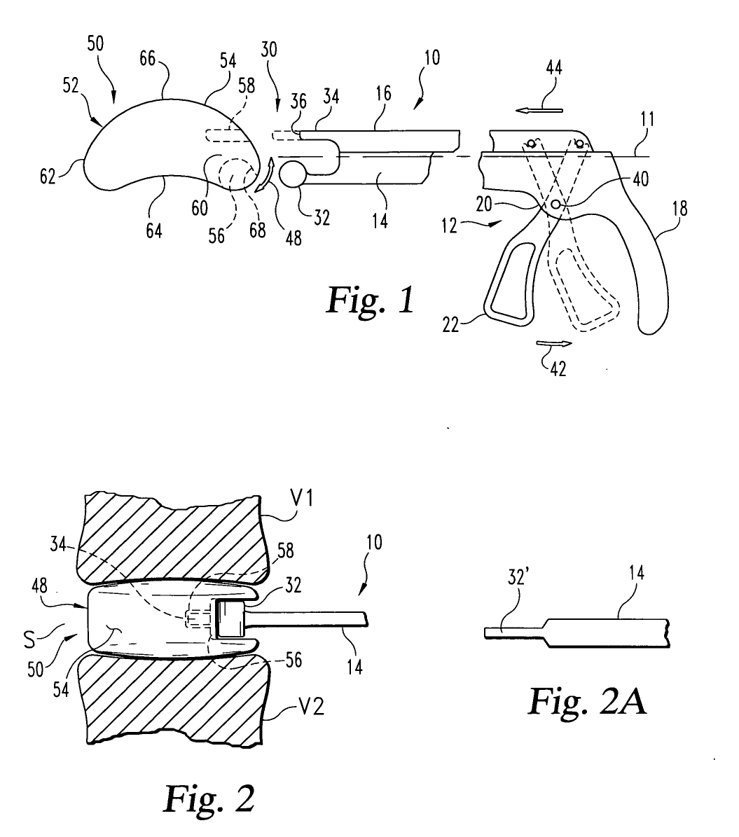

In FIG. 1 there is shown an assembly for spinal stabilization procedures that includes an inserter instrument 10 and a spinal implant 50. Inserter instrument 10 includes an actuator assembly 12 operable to remotely move an engaging assembly 30 at a distal end of actuator assembly 12. Engaging assembly 30 is engageable to implant 50 to provide an assembly that facilitates positioning and manipulation of implant 50 in a space between vertebrae.

Implant 50 includes a body 52 having a surface 54 and a first receptacle 56 and a second receptacle 58 in surface 54. Body 52 can have a concavo-convex shape as shown where one side 64 is concavely curved and the opposite side 66 is convexly curved. Opposite end portions 60, 62 having convexly curved outer end surfaces extend between the concave-convex sides. Receptacles 56, 58 can be formed adjacent one or both of the end portions 60, 62.

Other embodiments contemplate other shapes for implant 50, including circular shapes, spherical shapes, oval shapes, D-shapes, polygonal shapes, rectangular shapes, and irregular shapes. The implants can be solid, or include one or more cavities or chambers or the like to allow for bone growth. The implants can be made from one or more sections assembled prior to implantation or during implantation. The implant 50 can be made from any suitable material, including metals and metal alloys, polymers, ceramics, carbon fiber, and bone, for example. Receptacles 56, 58 are formed in the surface of the implant to receive the inserter instrument 10 for implantation of the implant into the space between vertebrae.

As shown in FIG. 2, implant 50 can be positioned in space S between vertebrae V1 and V2. Vertebrae V1 and V2 can be adjoining vertebrae of the spinal column, or vertebrae separated by one or more removed vertebrae and associated spinal discs. The vertebrae can comprise vertebrae of the sacral, lumbar, thoracic or cervical region of the spine.

Actuator assembly 12 of inserter instrument 10 includes a mounting member 14, a trigger 22, and a rail member 16 slidably mounted to mounting member 14. Rail member 16 and mounting member 14 extend distally from housing portion 20 and form a shaft assembly extending along a longitudinal axis 11. The proximal end of mounting member 14 includes a fixed handle member 18 extending proximally from intermediate housing portion 20. Trigger 22 is pivotally mounted to housing portion 20 and extends therein where an end of trigger 22 is linked to rail member 16. Pivoting of trigger 22 about mounting pin 40, as indicated by arrow 42, longitudinally and slidably displaces rail member 16 along mounting member 14 as indicated by arrow 44.

Engaging assembly 30 is provided at the distal end of mounting member 14 and rail member 16. Engaging assembly 30 includes a support member 32 at a distal end of mounting member 14 and a coupling member 34 at a distal end of rail member 16. Support member 32 includes a cylindrical shape, as shown in FIG. 2, with a circular cross-section, as shown in FIG. 1. Receptacle 56 can have a size and shape to accommodate placement of support member 32 therein.

Coupling member 34 forms an axial extension of rail member 16, and includes an elongated cylindrical shape extending to a distal end 36. Distal end 36 can be flat or include a rounded shape to facilitate insertion into receptacle 58. Receptacle 58 includes an elongated cylindrical shape along longitudinal axis 11 and conforms generally to the size and shape of coupling member 34. In a first supporting mode, support member 32 and thus instrument 10 can thus pivot in one plane relative to implant 50 as indicated by bi-directional arrow 48.

In use, support member 32 is positioned in receptacle 56 while coupling member 34 is displaced proximally, as shown in FIG. 1. The shape of support member 32 allows it to rotate in the first mode when positioned in receptacle 56 as indicated by arrow 48. Accordingly, inserter instrument 10 can be oriented in an initial insertion orientation relative to implant 50 that allows placement of support member 32 into receptacle 56. Inserter instrument 10 can then be pivoted as indicated by arrow 48 to align coupling member 34 with receptacle 58. In this orientation, lip 68 of end portion 60 extends transversely to longitudinal axis 11 and semi-constrains or retains support member 32 in receptacle 56 by resisting movement of implant 50 along longitudinal axis 11 and away from inserter instrument 10. In order to prevent implant 50 from pivoting about support member 32 and becoming disengaged therefrom, coupling member 34 is advanced distally as indicated by arrow 44 by pivoting trigger 22 as indicated by arrow 42 to the actuated position indicated by the dotted outline of trigger 22. This actuation in turn moves rail member 16 distally as indicated by the dashed line representation thereof and into receptacle 58, thus restraining the implant and preventing pivoting of implant 50 about support member 32 and rigidly securing it to engaging assembly 30 in the second mode. Implant 50 can then be positioned in the space between vertebrae and moved relative to the vertebrae to the desired location. Trigger 22 can then be released to move in the direction opposite arrow 42 to move rail member 16 in the direction opposite arrow 44 and remove coupling member 34 from receptacle 58.

Support member 32 includes a cylindrical shape extending in a first direction transversely to longitudinal axis 11 and coupling member 34 includes a cylindrical shape extending along longitudinal axis 11 and orthogonally or transversely to support member 32. Receptacles 56, 58 can include similarly situated and shaped receptacles that receive respecting ones of the support member 32 and coupling member 34. Receptacle 56 includes an opening in the surface of implant 50 that requires positioning of support member 34 into receptacle 56 with implant 50 obliquely oriented to longitudinal axis 11. Implant 50 can thereafter be pivoted into alignment along longitudinal axis 11 so that receptacle 58 is aligned to receive coupling member 34. In FIG. 2A, support member 32′ includes a cylindrical shape with a flat, disc shape in the first direction and a circular shape when viewed from the direction of FIG. 1. The flat disc shape can reduce the size of the receptacle into the implant.

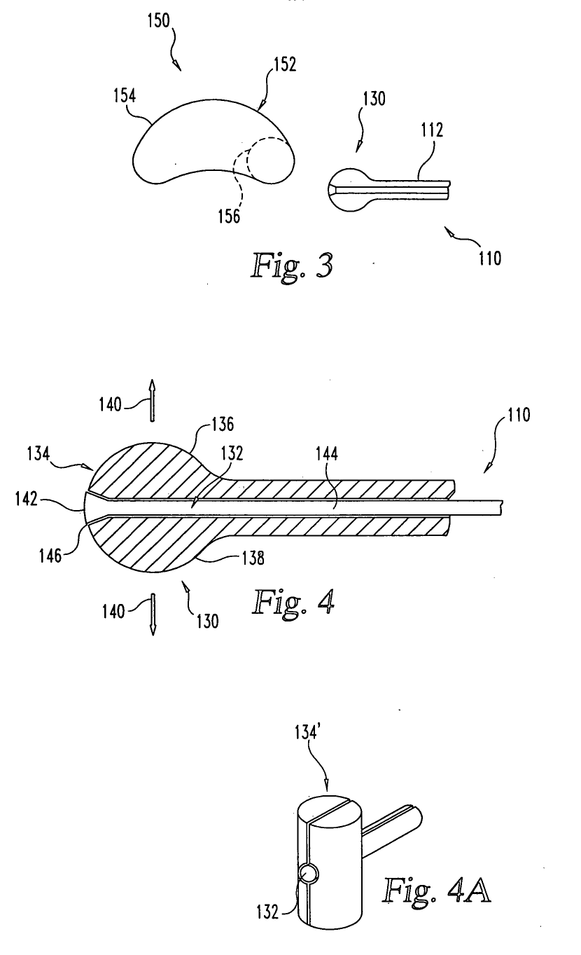

FIG. 3 shows another embodiment implant 150 and inserter instrument 110 that can be engaged to one another to provide an assembly for positioning implant 150 in a space between vertebrae. Implant 150 can be configured similar to implant 50 discussed above, but includes a single receptacle 156 formed in surface 152 of body 154. Other embodiments contemplate multiple receptacles for multiple implant engaging locations. Inserter instrument 110 includes an elongate shaft 112 and an engaging assembly 130 at a distal end of shaft 112. Engaging assembly 130 is positionable in receptacle 156 in a reduced size configuration and thereafter expandable in receptacle 156 to engage and grip implant 150 at the distal end of inserter instrument 110.

Engaging assembly 130 is shown in further detail in FIG. 4, and includes an actuating member 132 positioned in a coupling member 134. Coupling member 134 includes first and second coupling portions 136, 138 that are movable away from one another as indicated by arrows 140 to form an expanded configuration to grip implant 150. Actuating member 132 includes an enlarged end member 142 that is movable into a passage or space 146 between coupling portion 136, 138 to force coupling portions 136, 138 away from one another. In one form, the end member 142 includes a proximally tapered configuration extending to actuator shaft 144, and coupling portions 136, 138 form a distally opening and proximally tapered recess 146 to receive end member 142. In still outer embodiments, coupling member 134 may include three or more coupling portions.

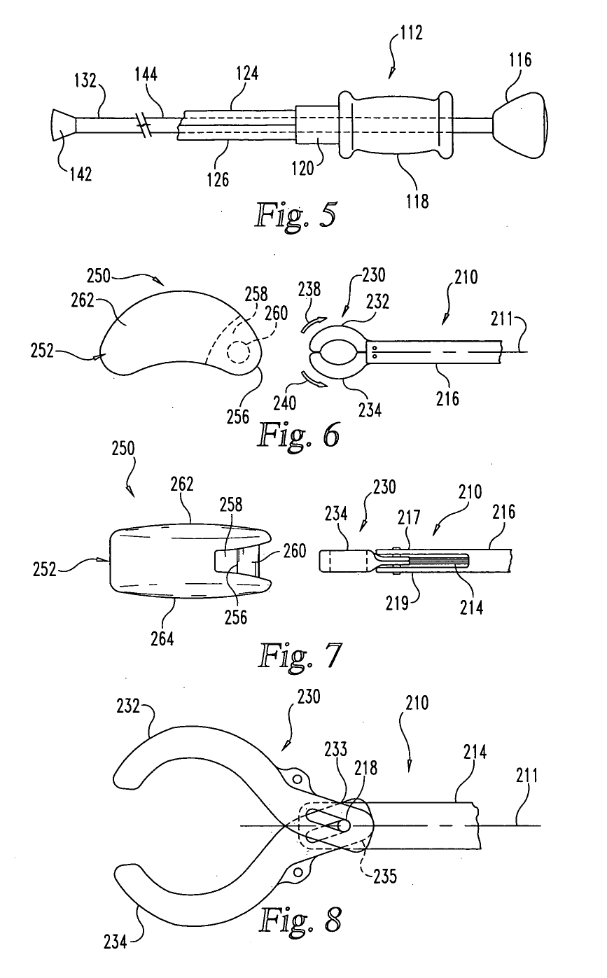

In FIG. 5, there is shown a proximal portion of one embodiment actuator assembly 112. Actuating member 132 is shown with coupling member 134 removed. Actuating member 132 includes proximally extending actuator shaft 144 positioned between coupling member shaft portions 124, 126. Shaft portions 124, 126 extend distally from an intermediate housing portion 120, and are integrally formed or otherwise engaged to housing portion 120 or to one another so that coupling member 134 is normally in an unexpanded configuration. Actuator shaft 144 extends proximally to a proximal knob or handle 116. Housing portion 120 and shaft portions 124, 126 are coupled to an intermediate handle 118.

In use, implant 150 is positioned with coupling member 134 in receptacle 156 in an unexpanded configuration. Actuating member 132 can be moved into engagement with coupling member 134 by proximally pulling, threading, or otherwise displacing actuator shaft 144 with handle 116 while holding handle 118. The expanded coupling member 134 contacts implant 150 in receptacle 156 and secures it to inserter instrument 110 for positioning in the space. To release the implant, handle 116 is moved distally to allow coupling portions 136, 138 to collapse and pass through the opening to receptacle 156. Other actuating arrangements are also contemplated, such as the trigger arrangement discussed above with respect to inserter instrument 10. Likewise, inserter instrument 10 can employ an actuator assembly like that discussed for inserter instrument 110.

When engaging assembly 130 is positioned in receptacle 156 in a reduced size configuration, it can also be partially expanded to semi-constrain implant 150 to inserter instrument 110 while allowing implant 150 and inserter instrument 110 to pivot relative to one another about coupling member 134 in a first mode. Accordingly, a wide range of angular orientations are possible that are defined by a cone extending outwardly from receptacle 156. Engaging assembly 130 can then be expanded with implant 150 and instrument 110 in any of these orientations to engage and rigidly constrain implant 150 at the distal end of inserter instrument 110 in a second mode.

In addition to a spherical shape, other shapes for coupling member 134 are contemplated. For example, FIG. 4A shows a coupling member 134′ that has a cylindrical shape extending along actuating member 132. The cylindrical shape can be divided into halves, thirds, fourths, etc. to provide portions movable away from one another to semi-constrain or rigidly constrain the implant to the inserter instrument when the coupling member is expanded in a correspondingly shaped receptacle of the implant.

Referring now to FIGS. 6 and 7, there is shown another implant 250 and an inserter instrument 210 engageable to implant 250 to provide an assembly for insertion of implant 250 into a space between vertebrae. Implant 250 can be configured similarly to implants 50, 150 discussed above, and can include a body 252 having a surface 254 and at least one receptacle 256 along surface 254. Receptacle 256 includes a passage 258 and a post 260 extending between upper and lower surfaces 262, 264 of implant 250. Other arrangements contemplate a post extending between sides of implant 250. In addition, post 260 can be side-loading as shown, or arranged to be top loaded or end loaded for engagement with the inserter instrument.

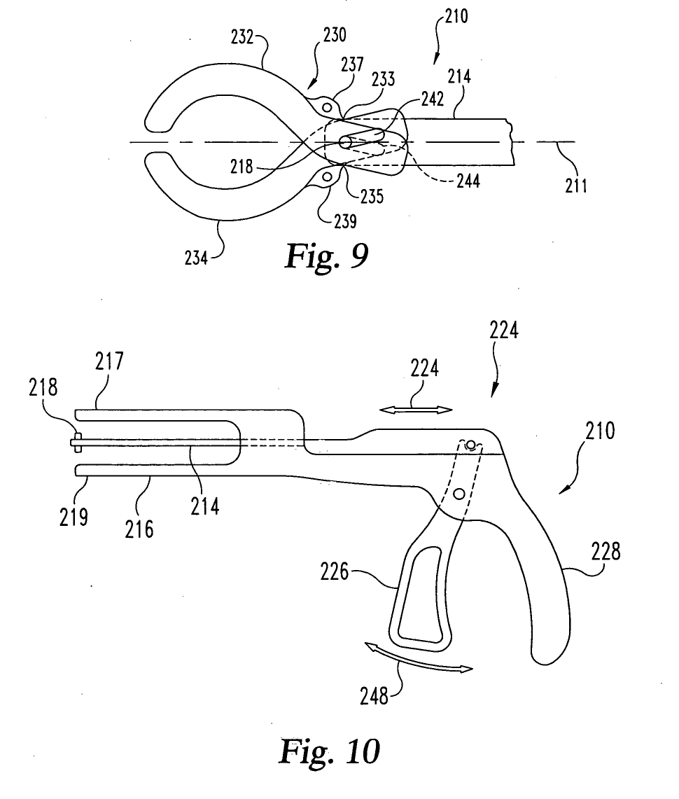

Inserter instrument 210 includes an actuating member 214 extending along a longitudinal axis 211 and includes a distal engaging assembly 230 engageable in receptacle 256 to secure implant 250 to inserter instrument 210. Engaging assembly 230 includes a first coupling member 232 and a second coupling member 234 pivotally coupled to one another along proximal arms 233, 235, respectively. Coupling members 232, 234 are movable away from one another, as indicated by arrows 238, 240, to an open position shown in FIG. 8 to allow placement of coupling members 232, 234 about post 260. Coupling members 232, 234 can then be closed to a coupling or closed position shown in FIGS. 6 and 9 to grip post 260 therebetween. Coupling members 232, 234 can include an arcuate shape to define a circular or rounded opening therebetween that is configured to tightly grip post 260 in the closed position. Non-circular shapes and engagement arrangements between the post and coupling members are also contemplated to resist relative rotation therebetween. In another embodiment, post 260 has a ball shape and coupling members 232, 234 form a socket-type arrangement that is grippable about the ball arrangement. In still other arrangements, the coupling members 232, 234 and the post 260 include teeth, ridges, or other features to provide interdigitation and secure rigidization when positioned in engagement with one another.

Referring further to FIGS. 8-10, coupling members 232, 234 are linked to actuating member 214, and can be pivotally mounted to respective ones of distal end arms 217, 219 of mounting member 216 at ears 237, 239 of coupling members 232, 234, respectively. Arm 233 includes a longitudinal slot 242 and arm 235 includes a longitudinal slot 244. Slots 242, 244 overlap one another with arm 233 and arm 235 in a scissors type arrangement. In the open position, an actuating pin 218 of actuating member 214 between arms 233, 235 is positioned into and adjacent proximal ends of slots 242, 244. Slots 242, 244 are obliquely oriented to longitudinal axis 211 and diverge distally away from one another in this configuration. As actuating member 214 is moved distally to close coupling members 232, 234, actuating pin 218 moves distally along slots 242, 244 and coupling members 232, 234 pivot about their respective coupling location to end arms 217, 219 of mounting member 216 until the distal ends of slots 242, 244 are aligned with pin 218 therein. Slots 242, 244 diverge proximally in the closed position.

In FIG. 10, there is shown actuator assembly 224 with a trigger 226 coupled to actuating member 214 and pivotally mounting to mounting member 214. Actuator assembly 224 also includes a proximal handle 228. Movement of trigger 226 toward handle 228 pivots trigger 226 about its mounting location with mounting member 216 as indicated by arrow 248 and distally displaces actuating member 214 as indicated by arrow 246. Actuator assembly 224 can be configured to bias coupling members 232, 234 to one of the open or closed positions, and can include a locking device to maintain coupling members in one or both of the open and closed positions.

In use, coupling members 232, 234 can be positioned around post 260 and loosely engaged thereto in a first mode to allow implant 250 to pivot relative to inserter instrument 210 to a desired orientation relative to longitudinal axis 211. Coupling members 232, 234 can then be tightly gripped to post 260 to secure implant 250 in position thereon in a second mode.

The above-described instruments and methods can be used in substantially open surgical procedures or in minimally invasive procedures that employ guide sleeves or tubes. Tubular retractors can provide greater protection to adjacent tissues, reduce the size of access incisions, provide direct visualization of the surgical site, and/or provide greater control of the method. The implants, instruments and methods may further be used in combination with disc space and endplate preparation and implant insertion through microscopic or endoscopic instruments that provide direct visualization of the surgical site.

The instruments discussed herein provide the surgeon the ability to control insertion of an implant into the space between vertebrae. The inserter instruments can facilitate positioning of the implant in the space along a non-linear insertion path. The inserter instruments can also be used to position multiple implants at various locations in the disc space, and also for insertion of one or more implants from other approaches to the disc space. Implants can be interbody fusion devices or cages that can be packed with bone growth material or other known substance and inserted into the space to promote bony fusion between vertebrae. Furthermore, the implants can have application for a disc prosthesis or a disc nucleus prosthesis that is to be inserted into the space between vertebrae.

While the invention has been illustrated and described in detail in the drawings and the foregoing description, the same is considered to be illustrative and not restrictive in character. All changes and modifications that come within the spirit of the invention are desired to be protected.

What is claimed is:

1. An assembly for spinal stabilization procedures, comprising:
an implant including a body with a first receptacle and a second receptacle;
an inserter instrument, comprising:
a proximal actuator assembly extending along a longitudinal axis;
an engaging assembly at a distal end of said proximal actuator assembly, said engaging assembly including a support member and a coupling member, said coupling member being movable relative to said support member along said longitudinal axis, said support member including a cylindrical body extending transversely to said longitudinal axis and said coupling member includes a cylindrical body extending along said longitudinal axis, said first and second receptacles each including a cylindrical size and shape to receive said respective first and second support members; and
wherein said implant is pivotal about said support member when received in said first receptacle and said coupling member prevents said implant from pivoting about said support member when received in said second receptacle simultaneously with said support member in said first receptacle.

2. The assembly of claim 1, wherein said cylindrical size and shape of said support member forms a disc.

3. The assembly of claim 1, wherein said implant includes an elongated convexly curved surface and an opposite elongated concavely curved surface extending generally along said longitudinal axis when engaged to said inserter instrument, said implant further including opposite convexly curved end surfaces extending between said concavely and convexly curved surfaces, said first and second receptacles extending into said body from one of said end surfaces.

4. The assembly of claim 1, wherein said actuator assembly includes a mounting member with a proximal handle, a trigger pivotally mounted to said mounting member, and a rail member movably mounted to said mounting member and linked to said trigger for movement by pivoting of said trigger.

5. The assembly of claim 4, wherein said support member extends from a distal end of said mounting member and said coupling member extends from a distal end of said rail member.

6. An assembly for spinal stabilization procedures, comprising:
an implant including a body with a receptacle;
an inserter instrument, comprising:
a proximal actuator assembly extending along a longitudinal axis; and
an engaging assembly at a distal end of said proximal actuator assembly, said engaging assembly including a coupling member and an actuating member extending through said coupling member, said actuating member being movable upon actuation of said actuator assembly to expand said coupling member in said receptacle and engage said implant to said engaging assembly.

7. The assembly of claim 6, wherein said coupling member is spherically shaped.

8. The assembly of claim 7, wherein said coupling member includes first and second coupling portions and said actuating member is positioned between said first and second coupling portions.

9. The assembly of claim 8, wherein said coupling portions include a distal recess and said actuating member includes a distal end member positioned in said recess when said coupling member is expanded.

10. The assembly of claim 9, wherein said end member and said recess are each tapered proximally along said longitudinal axis.

11. The assembly of claim 6, wherein said implant includes an elongated convexly curved surface and an opposite elongated concavely curved surface extending generally along said longitudinal axis when said implant is engaged to said inserter instrument, said implant further including opposite convexly curved end surfaces extending between said concavely and convexly curved surfaces, said receptacle including a spherical shape in one of said end surfaces.

12. The assembly of claim 6, wherein said coupling member is cylindrically shaped.

13. An assembly for spinal stabilization procedures, comprising:
an implant including a body with a receptacle and a post in said receptacle; and
an inserter instrument, comprising:
a proximal actuator assembly extending along a longitudinal axis; and
an engaging assembly at a distal end of said proximal actuator assembly engaged to said post, said engaging assembly including first and second coupling members at least one or which is movable relative to the other wherein distal ends of said coupling members are spaced in an open position for positioning around said post and movable with said actuator assembly to a closed position wherein said first and second coupling members are clampingly engaged to said post.

14. The assembly of claim 13, wherein said coupling members each include a concave curvature oriented toward one another to define a circular shaped opening therebetween in the closed position.

15. The assembly of claim 14, wherein said post includes a circular cross-section.

16. The assembly of claim 13, wherein said receptacle includes a passage extending around said post and said coupling members are positioned in said passage when positioned around said post in said closed position.

17. The assembly of claim 13, wherein said implant includes an upper surface and an opposite lower surface and said post extends in the direction between said upper and lower surfaces.

18. The assembly of claim 13, wherein said actuator assembly includes a mounting member extending along said longitudinal axis and said coupling members are pivotally mounted to a distal end of said mounting member.

19. The assembly of claim 18, wherein said actuator assembly includes an actuating member coupled to proximal ends of said coupling members, said actuating member being movable with actuation of said actuator assembly to move along said mounting member and move said coupling members between said open and close positions.

20. The assembly of claim 19, wherein said actuator assembly includes a proximal handle extending from said mounting member and a trigger pivotally coupled to said mounting member and linked to said actuating member, wherein pivoting of said trigger toward said handle longitudinally displaces said actuating member relative to said rail member.

21. The assembly of claim 13, wherein said implant includes an elongated convexly curved surface and an opposite elongated concavely curved surface extending generally along said longitudinal axis, said implant further including opposite convexly curved end surfaces extending between said concave and convex surfaces, said receptacle being formed at least one of said end surfaces and said post extending in said receptacle between opposite upper and lower surfaces of said implant in said receptacle.
